(19) United States
(12) Patent Application Publication
(10) Pub. No.: US 2021/0145862 A1
(43) Pub. Date: May 20, 2021

(54) HIGH MOLECULAR WEIGHT HYALURONIC ACID FOR ENHANCING EPITHELIAL SURVIVAL AND RECONSTITUTION OF BODY SURFACES

(71) Applicant: I.COM MEDICAL GMBH, MÜNCHEN (DE)

(72) Inventors: WOLFGANG GEORG KONRAD MÜLLER-LIERHEIM, MÜNCHEN (DE); GYSBERT-BOTHO VAN SETTEN, DANDERYD (SE)

(21) Appl. No.: 17/048,436

(22) PCT Filed: Apr. 17, 2019
(86) PCT No.: PCT/EP2019/059962

§ 371 Date: Oct. 16, 2020

Related U.S. Application Data
(60) Provisional application No. 62/659,180, filed on Apr. 18, 2018.

(51) Int. Cl. A61K 31/728; A61P 27/04; A61K 9/00

(52) U.S. Cl. CPC A61K 31/728; A61K 9/0048; A61K 9/0014; A61P 27/04

(57) ABSTRACT

The invention concerns a method for restoring or replenishing hyaluronic acid at an epithelial surface of a subject, comprising topically administering a composition to the epithelial surface of the subject, wherein the composition comprises high molecular weight hyaluronic acid, high molecular weight hyaluronic acid analogue, or a combination thereof, and wherein the epithelial surface has a deficiency in the amount or function of hyaluronic acid.

### CROSS-REFERENCE TO RELATED APPLICATION

The present application claims the benefit of U.S. Provisional Application Ser. No. 62/659,180, filed Apr. 18, 2018, which is hereby incorporated by reference herein in its entirety, including any figures, tables, nucleic acid sequences, amino acid sequences, or drawings.

### FIELD OF THE INVENTION

The present invention concerns the use of high molecular weight hyaluronic acid to replenish the epithelial surfaces of the body and compensate for the absence or dysfunction of naturally occurring polysaccharides.

### BACKGROUND OF THE INVENTION

Hyaluronic acid (HA) is produced at the surfaces of the human body by epithelial cells, among others. The presence of HA at surfaces can contribute considerably to the survival of the organism and the functional integrity of epidermal or other surface structures. HA is a critical component of extracellular matrix (ECM) of most vertebrate tissues (Vigetti D et al., “Hyaluronan Synthesis is Inhibited by Adenosine Monophosphate-activated Protein Kinase Through the Regulation of HAS2 Activity in Human Aortic Smooth Muscle Cells,” The Journal of Biological Chemistry, 2011, 286(10):7917-7924). Produced by specific synthases, mammalian cells can produce two specific HA synthases (HAS1 and HAS2) that produce high molecular weight HA (HMW-HA), in the range of millions of daltons (Da), whereas the other isoenzyme (HAS3) synthesizes HA of lower molecular mass HA in the range of several thousands of Da.

HA turnover is important for the maintenance of tissue homeostasis, and approximately 30% of HA is normally replaced by newly formed HA in a 24-hour period (Fox S B et al., “Normal Human Tissues, in Addition to Some Tumors, Express Multiple Different CD44 Isoforms, Cancer Res, 1994, 54:4539-46). Removal of HA can occur by endocytic uptake within the tissue, especially in lymph nodes and liver. The presence of reactive oxygen species (ROS) enhances HA turnover (Hrabarova E et al., “Pro-oxidative Effect of Peroxynitrite Regarding Biological systems: A Special Focus on High-Molar-Mass Hyaluronan Degradation,” Gen Physiol Biophys, 2011, 30:223-38; Hrabarova E et al., “Free-radical Degradation of High-Molar-Mass Hyaluronan Induced by Ascorbate Plus Cupric Ions: Evaluation of Antioxidative Effect of Cysteine-derived Compounds, Chem Biodivers, 2012, 9:309-17; and Soltes L et al. “Degradative Action of Reactive Oxygen Species on Hyaluronan Biomacromolecules,” 2006, 7:659-68).

The size of HA within tissue at a given time depends also on specific degrading enzymes (hyaluronidases) that can produce bioactive HA oligosaccharides. Synthesized in the cellular plasma membrane, increased levels of HA have been described in the presence of enhanced growth factor activity and cytokine presence such as during tissue regulation and wound healing, as well as during inflammation. Due to its invariant chemical structure, HA has a highly reduced likelihood of immunologic reactions, thus increasing its biocompatibility (Farwick M et al., “Fifty-kDa Hyaluronic Acid Upregulates Some Epidermal Genes Without Changing TNF-α Expression in Reconstituted Epidermis,” Skin Pharmacol Physiol, 2011, 24:210-217). An adverse reaction to externally applied HA is hence not to be expected. In adult tissues, HA synthesis is stimulated by injury, inflammation, and neoplastic tumors (Tammi R H et al., “Transcriptional and Post-Translational Regulation of Hyaluronan Synthesis,” FEBS J, 2011, 278(9):1419-28). In the synthesis by HAS, there are large differences between different cell types concerning the stimulants to which they respond. On the other hand, despite its simple structure, HA has a plethora of interacting properties with numerous proteins. Its capacity to interact with cell receptors, such as CD44 and receptor for HA-mediated cell motility (RHAMM), can trigger several responses (Misra S et al., “Interactions Between Hyaluronan and its Receptors (CD44, RHAMM) Regulate the Activities of Inflammation and Cancer,” Front Immunol, 2015, 6:201). The specific actions seem to be dependent on the molecular size.

The current understanding is that low molecular weight HA (LMW HA) has the most well known effects. Accordingly, the dermatology industry has favored LMW HA. Similarly, HA of ocular formulations intended for the surface of the eye is mainly LMW HA. High molecular weight HA (HMW HA) molecules have been shown to penetrate the surface of the skin and eye less effectively than LMW HA; consequently, HMW HA has thus far not been the main focus of interest for topical applications. If used, they are employed, for example, as lubricants at the surface of the eye, exploiting their properties as non-Newtonian fluids. However, in adult tissues, if HA synthesis is stimulated in injury, inflammation, and neoplastic tumors, then increased production of HA should contribute directly or indirectly to calming down those mediators such as cytokines and growth factors, which are recruited or activated by injury, inflammation, neoplastic tumors.

In conditions in which tissues are continuously stressed, either by predisposition or constant external challenge, they are subject to continuous enhanced production of HA. Over time, this could lead to a situation in which the tissue's regenerative capability and capacity for HA production are exceeded and/or exhausted. In these situations, the epithelium will suffer and the regulatory system for HA synthesis is out of balance. In HA's presence, the relatively constant expression of stimulators of HA expression may lead to their accumulation and to secondary effects. One of these secondary effects may be a pro-inflammatory state, such as that found in atopy.

### BRIEF SUMMARY OF THE INVENTION

In a healthy subject, and in youth, the production of hyaluronic acid (HA) usually copes with the demand of normal cell turnover and protection against the environment. In other conditions, however, such as disease, or in aged subjects, the quantity or quality of HA produced is not sufficient to maintain the integrity of tissue function at epithelial surfaces of the body. The inventors propose that topical application of high molecular weight (HMW) HA to epithelial surfaces of atopic and other subjects can serve to replenish or restore HA at the epithelial surface, protecting the epithelium from the deleterious effects that accompany a dearth of HA, or impaired HA, at the epithelial surface.

The average molecular weight of HA in the extracellular matrix (ECM) of healthy epithelium is 3 to 4 MDa, and subject to catabolism (30%-50% turnover per day, depending upon tissue type). From the fifth decade in life, the amount of free, not cell-bound HA in the extracellular space of the epithelium decreases dramatically, but the total amount of HA is not significantly changed; this is reported to be the main reason for parchy skin observed in elderly patients and is attributed, partly, to loss of repair ability and loss of water-binding capacity of the skin.

It may be that the average molecular weight of HA in the ECM of the skin is changing with age (e.g., because the ability of the cells to synthesize HA decreases with age and thus the average molecular weight becomes lower). All of these effects are not directly related to the molecular weight-dependent signal function of HA described in the literature. It is possible that the mechanism of neovascularization is controlled by the mechanical stability of epithelia and perhaps other tissues. This then would be an elegant explanation as to why HMW HA acts anti-angiogenetically by simply ensuring a stable ECM and low molecular weight HA acts angiogenically by simply shifting the equilibrium of the molecular weight of HA in the ECM to lower values, thus decreasing the mechanical stability of the tissue. The inventors propose that a stable ECM down-regulates inflammation and contributes to prevent inflammation to enter into a chronic stage; moreover, this role of HA in the matrix may help to interpret and understand the controversial reports of the role of HA in the progression of tumors.

The invention concerns compositions and methods for use in restoring HA at an epithelial surface of a human or non-human subject. The compositions comprise HMW HA, HMW HA analogue, or a combination thereof, and the restorative methods comprise topically administering the composition to an epithelial surface having a deficiency in the amount or function of HA.

In some embodiments, the method reduces the severity, or delays the onset, of a hypersensitivity reaction at an internal or external epithelial surface of the body of a human or non-human animal subject. By topically administering a composition containing a very high molecular weight form of hyaluronic acid to a body surface, such as skin or a mucous membrane, the invention can attenuate or delay the onset of the hypersensitivity reaction at the epithelial surface. The composition may be topically administered to the epithelial surface before, during, and/or after onset of the hypersensitivity reaction at the epithelial surface.

Without being limited by theory of mechanism of action, it is proposed that when topically administered to a surface of epithelium, the composition can interfere with the binding of pro-inflammatory cytokines with receptors at the epithelial surface, thereby improving cellular stability and integrity.

### DETAILED DESCRIPTION OF THE INVENTION

Hyaluronic acid (HA) is a carbohydrate—a mucopolysaccharide, specifically, which can be found in living organisms. The biological functions of endogenous HA include maintenance of the elastoviscosity of liquid connective tissues such as joint synovial fluid and eye vitreous fluid (Necas J et al., “Hyaluronic acid (hyaluronan): a review”, Veterinarni Medicina, 2008, 53(8):397-411; Stern R et al., “Hyaluronan fragments: An information-rich system”, European Journal of Cell Biology, 2006, 85:699-715). Although the specific mechanisms involved in the diverse signaling of HA are still poorly understood, it is known that HA can modulate multi-faceted biological effects that can vary depending on HA size (Cyphert J M et al., “Size Matters: Molecular Weight Specificity of Hyaluronan Effects in Cell Biology,” International Journal of Cell Biology, 2015, Epub 2015 Sep. 10, 563818).

Sodium hyaluronate and other viscoelastic agents have been used in intraocular surgery since the 1970s and for treatment of dry eyes since the 1980s (Higashide T and K Sugiyama, “Use of viscoelastic substance in ophthalmic surgery—focus on sodium hyaluronate,” Clinical Ophthalmology, 2008, 2(1):21-30; Polack FM and MT McNiece, “The treatment of dry eyes with Na hyaluronate (Healon)—preliminary report, 1982, 1(2):133-136); however, little attention has been paid thus far to the biological function of hyaluronic acid in epithelia (Müller-Lierheim WGK, “Tränenersatzlösungen, Neues über Hyaluronsäure,” Aktuelle Kontaktologie, April 2015, 17-19).

The HA used in the compositions and methods of the invention is high molecular weight (HMW). In some embodiments, the HMW HA has an intrinsic viscosity of greater than 2.5 m³/kg. Furthermore, in cases in which the epithelial surface is epithelium of the ocular surface, the concentration of HMW HA and/or HMW HA analogue is preferably <0.2% w/v. In other cases, in which the epithelial surface is epithelium of the skin, for example, the concentration of HMW HA and/or HMW HA analogue is preferably within the range of 0.2% to 3.0% w/v. The intrinsic viscosity may be determined by the method of the European Pharmacopoeia 9.0, “Sodium Hyaluronate”, page 3584 (which is incorporated herein by reference in its entirety). Briefly, the intrinsic viscosity [η] is calculated by linear least-squares regression analysis using the Martin equation: Log₁₀(nᵣ−1/c)=log₁₀[η]+κ[η]c. In some embodiments, the hyaluronic acid has an intrinsic viscosity of greater than 2.9 m³/kg.

In some embodiments, the compositions used in the invention have a hyaluronic acid concentration of 0.1 to 0.19% w/v.

In some embodiments, the compositions used in the invention have: a) a pH of 6.8-7.6; b) an osmolarity of 240-330 mosmol/kg; c) a NaCl concentration of 7.6-10.5 g/l; and/or d) a phosphate concentration of 1.0-1.4 mmol/l.

In some embodiments the composition used in the invention is a clear and colourless solution, free from visible impurities. It is envisaged that the composition is sterile.

In some embodiments the composition used in the methods and kits of the invention is Comfort Shield® preservative-free sodium hyaluronate eye drops.

In some embodiments, the HA has a molecular weight of at least 3 million Daltons as calculated by the Mark-Houwink equation. In some embodiments, the HA has a molecular weight in the range of 3 million to 4 million Daltons as calculated by the Mark-Houwink equation.

In some embodiments, the HMW HA is hyaluronan. In some embodiments, the HMW HA is cross-linked, such as hylan A and hylan B. In some embodiments, the HMW HA is non-cross-linked. In some embodiments, the HMW HA is linear. In some embodiments, the HMW HA is non-linear. In some embodiments, the HMW HA is a derivative of hyaluronan, such as an ester derivative, amide derivative, or sulfated derivative, or a combination of two or more of the foregoing.

The composition may be in the form of a liquid, solid, or semi-solid. In some embodiments, the composition is a liquid (e.g., fluid, spray, lotion, aerosol). In some embodiments, the composition is a solid (e.g., tablet, capsule, granule, powder, sachet, dry powder inhalant, chewable). In some embodiments, the composition is a semi-solid (e.g., cream, ointment, gel, jelly, paste, salve, balm, mousse, foam, transdermal patch, suppository).

In some embodiments, the epithelial surface to which the composition is topically administered is deficient in the amount of HA or function of HA at the time the composition is topically administered. In other embodiments, the epithelial surface of the subject to which the composition is topically administered is not deficient in the amount of HA or function of HA at the time the composition is topically administered.

In some embodiments, the epithelial surface to which the composition is topically administered is that of skin. In some embodiments, the epithelial surface to which the composition is topically administered is the epithelial surface of a mucous membrane such as mucosa of the eye, ear, gastrointestinal tract (e.g., mouth, esophagus, stomach, small intestine, large intestine, colon, cecum, rectum, or anus), respiratory tract (e.g., nose, larynx, trachea, bronchial tree, lung alveoli), or urogenital tract (e.g., urinary bladder, ureter, urethra, kidneys, vas deferens, vulva, vagina, cervix, uterus, Fallopian tubes).

In some embodiments, the epithelial surface to which the composition is topically administered is an ocular surface (e.g., conjunctiva). In some embodiments, the epithelial surface to which the composition is topically administered is a non-ocular surface.

The administered composition can increase or enhance the visual performance of an eye to which it is administered, whether or not the ocular surface has diminished HA or HA function at the time the composition is administered to the eye. The administered composition helps to stabilize the fluid film of the eye (e.g., the tear film at the ocular surface), optimizing vision and visual performance, which is particularly beneficial in extreme conditions such as aviation and other settings in which it is impossible or undesirable to blink, space travel, diving in polluted or intoxicated fluids, or operating in areas with extreme climates such as cold, heat, and dryness.

An increase or enhancement of visual performance can be defined as an increase or enhancement in the speed and/or accuracy of processing visual information. For example, visual performance can be described as how quickly and accurately an individual can process visual stimuli that are defined in terms of criteria such as adaptation luminance, target contrast, and target size. Methods for assessing visual performance and changes in visual performance are known in the art (see, for example, Toda I et al., “Visual performance after reduced blinking in eyes with soft contact lenses or after LASIK,” J Refract. Surg., 2009, Jan., 25 (1):69-73; and Rea M S and M J Quellette, “Relative visual performance: A basis for application,” Lighting Res. Technol., 1991, 23(3):135-144, which are incorporated herein by reference in their entirety).

The composition may be administered as a fluid to the ocular surface of one or both eyes of the subject by any topical administration method. For example, the fluid may be administered as one or more drops from a device for dispensing eye drops, such as an eye dropper. The fluid may be self-administered or administered by a third party. The dosage administered, as single or multiple doses, to an ocular surface will vary depending upon a variety of factors, including patient conditions and characteristics, extent of symptoms, concurrent treatments, frequency of treatment and the effect desired. For example, one or more drops (of, for example, about 30 microliters each) may be administered. Typically, administration of 1-3 drops, one to three times per day, will be sufficient, particularly for acute ocular surface inflammation conditions. In cases of chronic ocular surface inflammation, however, more frequent administration may be needed, particularly during the initial phase of treatment, e.g., 1-3 drops for four, five, six, seven, eight, nine, ten, or more times per day.

Advantageously, in some embodiments, the frequency of administration of the composition and/or the amounts per dose can be decreased with time, as HA at the epithelial surface is preserved or restored. For example, in some cases, after four weeks, the amount administered may be reduced and/or the frequency of administrations each day may be reduced or the frequency of administrations may be reduced to semi-daily.

The composition may be administered prophylactically, before a condition exists, e.g., before a hypersensitivity reaction develops, to reduce the severity of the condition and/or delay its onset; or the composition may be administered therapeutically, after a condition, such as a hypersensitivity reaction, exists, to reduce the severity of the condition. Optionally, the composition is administered prophylactically before an event or stimulus occurs that causes the condition, such as trauma (e.g., non-surgical trauma), surgery, infection (e.g., exposure to bacterial, viral, fungal, protozoan (e.g., amoeba)), or exposure to an antigen that causes hypersensitivity reaction in the subject. In some embodiments, onset of the condition is delayed indefinitely (i.e., prevented). In some embodiments, one or more symptoms of the condition are alleviated or eliminated. In some embodiments, all symptoms of the condition are alleviated or eliminated.

The composition may also be administered prophylactically to subjects that are particularly susceptible or prone to infection. The subject's immunocompromised condition may have one or more causes, such as a medical treatment (e.g., radiation therapy, chemotherapy or other immunosuppressing treatment), environmental exposure (e.g., radiation exposure), or genetic defect.

In cases in which the condition exists at the time of composition administration and the composition is administered therapeutically, optionally, the method further comprises the step of identifying the subject as having the condition prior to administering the fluid.

In some embodiments, the condition may have one or more of the following characteristics: leukocyte invasion at the ocular surface and tears, CD44 upregulation at the ocular surface, and activation of an immune cascade that includes one or more of IL-1, IL-2, IL-5, IL-6, IL-8, CXCL8, IL-10, IL-12, IL-16, IL-33, MCPJ, CCL2, MIP1d (also known as CCL15), ENA-78, CXCLS, sILR1, sIL-6R, sgp sEGFR, sTNFR, I-17A, IL-21, IL-22, CXCL9, MIG, CXCL11, I-TAC, CXCL10, IP-10, MIP-1β, CCL4, RANTES, and CCL5.

The condition may be caused by various stimuli—external, internal, or both. In some embodiments, the condition is caused by an external stimulus resulting in a disruption of the smoothness and/or integrity of the epithelium of the ocular surface (e.g., medical therapy, ocular surgery, non-surgical trauma, contact lens wearing, microbial infection, allergen, hapten, toxic agent, or irritative substance).

Various medical therapies, such as small molecule pharmaceuticals, radiation such as UV and radio-therapy, and biologics, may cause the condition. For example, the condition may be caused by a “beta blocker”, which refers to agents that inhibit or block the activity of one or more beta-adrenergic receptors. Beta blockers may be used for treatment of hypertension, stable and unstable angina, arrhythmias, migraine, bleeding esophageal varices, heart failure, and coronary artery disease, among other indications. Some beta-blockers antagonize one specific subtype of beta-adrenergic receptors (e.g., a beta-1 selective beta blocker which selectively antagonizes the beta-1 adrenergic receptor), whereas other beta-blockers are non-selective. Some beta-blockers can inhibit the effect of ligands such as noradrenaline or norepinephrine on one or more beta-adrenergic receptors. Accordingly, the term “beta-blocker” refers to all types of antagonists or inhibitors of beta-adrenergic receptors, regardless of whether the beta-blocker antagonizes one, two or more beta-adrenergic receptors and regardless of whether they affect other processes. Examples of beta-blockers include, but are not limited to, acebutolol, alprenolol, atenolol, betaxolol, bisoprolol, bopindolol, bucindolol, butaxamine, carteolol, carvedilol, celiprolol, esmolol, labetalol, levobunolol, medroxalol, metipranolol; metoprolol, nadolol, nebivolol, nadolol, oxprenolol, penbutolol, pindolol, propafenone, propranolol, sotalol, timolol, and eucommia bark.

In some embodiments, the condition is allergy of the eye. In some embodiments, the condition is non-infectious keratoconjunctivitis caused by an external damage, allergic keratoconjunctivitis (such as seasonal allergic keratoconjunctivitis), or infectious keratoconjunctivitis such as viral keratoconjunctivitis, bacterial conjunctivitis, fungal keratoconjunctivitis, and parasitic conjunctivitis. In some embodiments, the condition is caused by an internal stimulus (e.g., hormonal disturbance (such as menopause and andropause), rheumatic disease, epithelial-mesenchymal transition (EMT), or autoimmune disease).

The condition may be caused by a wound in the epithelium of the eye. In some embodiments, the wound is caused by physical trauma, chemical trauma, laser treatment such as excimer, or radiation (radiation injury). In some embodiments, the wound is caused by an ocular surgery. Examples of ocular surgeries include but are not limited to natural or artificial corneal transplantation, corneal implantation (e.g., intracorneal rings (ICRs), and keratoprosthesis), glaucoma surgery, cataract surgery (e.g., phacoemulsification, extracapsular cataract surgery, or intracapsular surgery), refractive surgery (e.g., radial keratotomy or refractive corneal incision), retinal surgery, squint (strabismus) surgery, corrective laser eye surgery (e.g., laser-assisted in situ keratomileusis (LASIK) or photorefractive keratectomy (PRK)), and cross-linking surgery. Administration of the fluid of the invention before, during, and/or after ocular surgery, such as glaucoma surgery, can improve clinical outcome, for example, by accelerating recovery, including recovery of visual performance after surgery, reducing scarring, and reducing itching, irritation, pain, and other discomfort.

The fluid may be administered to reduce or prevent or delay onset of ocular discomfort such as itchiness or ocular pain. The pain may have one or more causes. For example, the ocular pain may be pain associated with mechanical, chemical, or thermal stimulation of the ocular surface. The ocular pain may be associated with acute or chronic inflammation, or immune response. With the reduction of the pain, comes the reduction of secondary neuroinflammatory effects (Belmonte C et al., “TFOS DEWS II pain and sensation report”, The Ocular Surface, 15:404-437). The cause of the pain may be known or unknown.

### Atopy

In some embodiments, the subject to which the HMW HA composition is topically administered has atopy. Normally, the immune system, which includes antibodies, defends the body against foreign substances called antigens. However, in susceptible people, the immune system can overreact when exposed to certain substances (allergens).

Allergy is the result of hypersensitivity reactions and can be immediate or delayed. Classical immunoglobulin E (IgE)-dependent reactions are involved, such as asthma, conjunctivitis, allergic rhinitis, atopic eczema allergic urticarial and even anaphylaxia. It is the propensity of an individual to produce IgE antibodies in response to various antigens in the individual's environment that leads to the establishment and enhanced predisposition to develop immediate hypersensitivity that is called atopy. Atopy is not the same as allergy. Both are linked to inflammation, but atopy does not follow the inflammatory cascade and does involve pro-inflammatory regulators. Links to allergy do exist; however, atopy is the predisposition to exhibit an excessive immunologic response (e.g., in the form of a hypersensitivity reaction), whereas allergy is the direct reaction to an allergen. According to Brown M A and J M Hanifin, “Atopic Dermatitis”, Current Opinion in Immunology, 2(4):531-534, interleukin-4 (IL-4) may be particularly important in human IgE synthesis and may have an important role in controlling mast cells and IgE production in atopic dermatitis. Allergic (including atopic) and other hypersensitivity disorders are inappropriate or exaggerated immune reactions to foreign antigens. Inappropriate immune reactions include those that are misdirected against intrinsic body components, leading to autoimmune disease.

The skin, as an interface between the organism and the external environment, plays a critical role in protecting and supporting the life it encloses. Importantly, in conditions of atopy, there is an associated dysfunction of the epidermal barrier (Brown S and N J Reynolds, “Atopic and non-atopic eczema”, BMJ, 2006, 332:584). On the skin, atopy is visible as atopic eczema, which is an itchy inflammatory skin condition. Atopy is typically associated with less tight junction between epithelial cells, which makes the body surfaces more vulnerable to the environment. Without being limited by theory of mechanism of action, topical administration of a composition comprising HMW HA can contribute to the strengthening of the ECM and thus help to protect the body surfaces of atopic patients.

Hypersensitivity reactions are divided into four types by the Gell and Coombs classification. Hypersensitivity disorders often involve more than one type. Type I reactions (immediate hypersensitivity) are IgE-mediated. Antigen binds to IgE that is bound to tissue mast cells and blood basophils, triggering release of preformed mediators (e.g., histamine, proteases, chemotactic factors) and synthesis of other mediators (e.g., prostaglandins, leukotrienes, platelet-activating factor, cytokines). These mediators cause vasodilation, increased capillary permeability, mucus hypersecretion, smooth muscle spasm, and tissue infiltration with eosinophils, type 2 helper T (T_{H}2) cells, and other inflammatory cells. Type I reactions typically develop less than one hour after exposure to antigen. Type I hypersensitivity reactions underlie all atopic disorders (e.g., allergic asthma, rhinitis, conjunctivitis) and many allergic disorders (e.g., anaphylaxis, some cases of angioedema, urticarial, latex and some food allergies). The terms atopy and allergy are often used interchangeably but, as indicated above, are different. Atopy is an exaggerated IgE-mediated immune response; all atopic disorders are type I hypersensitivity disorders. Allergy is any exaggerated immune response to a foreign antigen, regardless of mechanism. Thus, all atopic disorders are considered allergic, but many allergic disorders (e.g., hypersensitivity pneumonitis) are not atopic. Allergic disorders are the most common disorders among people.

Atopic disorders most commonly affect the nose, eyes, skin, and lungs. These disorders include conjunctivitis, extrinsic atopic dermatitis, immune-mediated urticarial, immune-mediated angioedema, acute latex allergy, some allergic lung disorders (e.g., allergic asthma, IgE-mediated components of allergic bronchopulmonary aspergillosis), allergic rhinitis, and allergic reactions to venomous stings.

The subject to which the HMW HA composition is administered may have a genetic risk factor associated with atopy or allergic disease, such as a predisposing gene or variation (Portelli M A et al., “Genetic risk factors for the development of allergic disease identified by genome-wide association”, Clinical & Experimental Allergy, 2014, 45:21-21; Tamari M et al., “Genome-wide Association Studies of Atopic Dermatitis”, Journal of Dermatology, 2014, 41:213-220; Hinds D A et al., “A Genome-Wide Association Meta-Analysis of Self-reported Allergy Identifies Shared and Allergy-Specific Susceptibility Loci”, Nat Genet, 2013, 45(8): 907-911; Bønnelykke, K et al.,

“Meta-analysis of genome-wide association studies identifies ten loci influencing allergic sensitization”. Nature Genetics, 2013, 45 (8): 902-906; Saunders S P et al., “Tmem79/Matt is the matted mouse gene and is a predisposing gene for atopic dermatitis in human subjects,” J Allergy Clin Immunol, 2013, 132(5): 1121-1129. In some embodiments, the subject has a predisposing gene or genetic variation (e.g., a polymorphism) at a locus associated with atopy, such as C11orf30, STAT6, SLC25A46, HLA-DQB1, IL1RL1/IL18R1, TLR1/TLR6/TLR10, LPP, MYC/PVT1, IL2/ADAD1, HLA-B/MICA, Tmem79/Matt, or a combination of two or more of the foregoing.

### Keratoconus

In some embodiments, the subject to which the HMW HA composition is topically administered has keratoconus (KC). Keratoconus is a progressive eye disease in which the normally round cornea thins and begins to bulge into a cone-like shape. This cone shape deflects light as it enters the eye on its way to the light-sensitive retina, causing distorted vision. Keratoconus can occur in one or both eyes and often begins during a person's teens or early 20s.

As the cornea becomes more irregular in shape, it causes progressive nearsightedness and irregular astigmatism to develop, creating additional problems with distorted and blurred vision. Glare and light sensitivity also may occur.

Often, keratoconic patients experience changes in their eyeglass prescription every time they visit their eye care practitioner.

On the surfaces such as the skin, age presents itself with dryness, decrease of elasticity, and the accompanying complaint of itching. Itching and local irritation are one of the hallmarks of atopy. On the eye, it can provoke eye rubbing. Eye rubbing again is considered a riskfactor (Galvin V et al., “Keratoconus: An Inflammatory Disorder?”, Eye, 2015, 29:843-859) as it leads to disturbance of cytokine balance at the ocular surface, which can be one of the key risk factors of KC development (Balasubramanian S A et al., “Effects of Eye Rubbing on the Levels of Protease, Protease Activity, and Cytokines in Tears: Relevance in Keratoconus,” Clin Exp Optom, 2013, 96(2):214-218). As indicated above, IL-4 could be particularly important in human IgE synthesis and may have an important role in controlling mast cells and IgE production in atopic dermatitis. Here, the use of HA could reduce the severity of KC (Kolozsvári B L et al., “Association Between Mediators in the Tear Fluid and the Severity of Keratoconus”, Ophthalmic Res., 2014, 51:46-51). The pathophysiology of KC is currently not considered directly inflammatory in nature but quasiinflammatory, i.e., inflammation-related or associated (McMonnies C W, “Inflammation and Keratoconus”, Optometry and Vision Science, February 2015, 92(2):e35-e41), involving specific subclinical inflammatory processes (Lema I et al., “Inflammatory Molecules in the Tears of Patients with Keratoconus”, Ophthalmology, 2005,112:654-659; and Lema I et al., “Subclinical Keratoconus and Inflammatory Molecules from Tears”, Br J Ophthalmol, 2009; 93:820-824). This process involves proinflammatory mediators in tear film such as IL-6 and MMP-9 (Jun A S et al., “Subnormal Cytokine Profile in the Tear Fluid of Keratoconus Patients,” PLoS One, 2011; 6:1-8). In parallel to corneal KC, fibroblasts show increased binding capacity for IL-1 (Fabre E J et al. “Binding Sites for Human Interleukin 1 Alpha, Gamma Interferon and Tumor Necrosis Factor on Cultured Fibroblasts of Normal Cornea and Keratoconus,” Curr Eye Res, 1991; 10:585-592), suggesting a link to inflammation. In the progress of KC, initial changes in the cornea stroma may trigger the onset of contour anomaly. Increased anomaly will then trigger increased stress on the corneal surface. Here, the epithelium plays a key role in the maintenance of the cytokine homeostasis of the surface and KC corneas have increased IL-1α and IL-1β expression (Zhou L et al., “Expression of Wound Healing and Stress-Related Proteins in Keratoconus Corneas, Curr Eye Res, 1996, 15:1124-1131; Bosnar D et al., “Influence of Interleukin-1α and Tumor Necrosis Factor-a Production on Corneal Graft Survival”, Croat Med J, 2006, 47(1):59-66; and Pearson A R et al., “Does Ethnic Origin Influence the Incidence or Severity of Keratoconus?,” Eye (Lond), 2000; 14(Pt 4):625-628). Corneal epithelium has been shown to secrete IL-1 after injury or tissue damage as well as after apoptosis (Wilson S E et al., “Epithelial Injury Induces Keratocyte Apoptosis: Hypothesized Role for the Interleukin-1 System in the Modulation of Corneal Tissue Organization and Wound Healing, Exp Eye Res, 1996, 62(4):325-327). In general, IL-1α is upregulated not only during inflammation but also by corneal trauma (West-Mays J A et al., “Repair Phenotype in Corneal Fibroblasts is Controlled by an Interleukin-1α Autocrine Feedback Loop”, Investigative Ophthalmology & Visual Science, June 1997, 38(7):1367-1379). At the same time fibroblasts from KC patients show elevated expression for IL-1α receptors (Bureau J et al., “Modification of Prostaglandin E2 and Collagen Synthesis in Keratoconus Fibroblasts Associated with an Increase of Interleukin 1 Alpha Receptor Number,” C R Acad Sci III, 1993, 316:425-430). Another key actor may be IL-17, a proinflammatory cytokine that is associated with many chronic inflammatory conditions ( ). Interestingly, Jun et al. (2011) detected elevated levels of IL-17 in tear fluid samples of KC patients. IL-17 has been associated with pathogenic mechanisms in corneal inflammation by stimulating stromal cells to secrete various pro-inflammatory cytokines (Maertzdorf J et al., “IL-17 Expression in Human Herpetic Stromal Keratitis: Modulatory Effects on Chemokine Production by Corneal Fibroblasts,” J Immunol, 2002 Nov. 15, 169(10):5897-5903) including IL-6, IL-8, and intercellular adhesion molecule 1 (ICAM-1) (Gabr M A et al., “Interleukin-17 Synergizes With IFNγ or TNFα to Promote Inflammatory Mediator Release and Intercellular Adhesion Molecule-1 (ICAM-1) Expression in Human Intervertebral Disc Cells,” J Orhop Res, 2011, 29(1):1-7). As stated above, despite its simple structure, HA has a plethora of interacting properties with numerous proteins (Vigetti D et al., “Hyaluronan Synthesis is Inhibited by Adenosine Monophosphate-activated Protein Kinase Through the Regulation of HAS2 Activity in Human Aortic Smooth Muscle Cells,” Journal of Biological Chemistry, 2011, 286(10):7917-7924). As stated by Vignetti et al. (2011), HMW HA (>1000 kD) and CD44 induce cell migration and promote the wound healing process (Tzircotis G et al., “Chemotaxis Towards Hyaluronan is Dependent on CD44 Expression and Modulated by Cell Type Variation in CD44-Hyaluronan Binding”, Journal of Cell Science, 2005, 118(21):5119-5128); moreover, the directionality of cell migration is strongly dependent on CD44 expression and on the HA gradient in extracellular matrix (ECM) environment (Acharya PS et al., Fibroblast Migration is Mediated by CD44-Dependent TGF Beta Activation,” J Cell Sci, 2008, 121(Pt 9):1393-1402)). Other engagement of HA include regulation and binding of growth factors and regulation of enzymatic activities.

As atopy is a risk factor for the development of KC, as prophylaxis to prevent or delay the onset of KC, the composition may be topically administered to the ocular surface of atopic patients under 30 year of age, and in all young contact lens wearers, because contact lens wearing causes corneal tissue changes similar to atopy.

Optionally, the HMW HA may be topically administered to the ocular surface before, during, or after one or more treatments for KC, such as: corneal cross-linking (CXL), custom soft contact lenses, gas permeable contact lenses, “piggybacking” contact lenses, hybrid contact lenses, scleral and semi-scleral lenses, prosthetic contact lenses, surgically applied corneal inserts (e.g., Intacs products), topography-guided conductive keratoplasty, or corneal transplant, or a combination of two or more of the foregoing.

### Additional Components

Optionally, the composition further includes one or more bioactive agents (e.g., a hydrophobic active ingredient). As used herein, the term “bioactive agent” refers to any substance that has an effect on the human or non-human animal subject when administered in an effective amount to affect the tissue. The bioactive agent may be any class of substance such as a drug molecule or biologic (e.g., polypeptide, carbohydrate, glycoprotein, immunoglobulin, nucleic acid), may be natural products or artificially produced, and may act by any mechanism such as pharmacological, immunological, or metabolic. Examples of classes of bioactive agents include substances that modify the pressure of the eye (e.g., enzyme inhibitors) and anti-angiogenic agents. Some specific examples of bioactive agents include steroids (e.g., corticosteroids), antibiotics, immunosuppressants, immunomodulatory agents, tacrolimus, plasmin activator, anti-plasmin, and cyclosporin A. In some embodiments, the bioactive agent is a steroid or antibiotic to treat or prevent eye infection; glaucoma drug such as prostaglandin analog, beta blocker, alpha agonist, or carbonic anhydrase inhibitor; agent for allergy eye relief such as histamine antagonist or non-steroidal anti-inflammatory drug; or mydriatic agent. Unfortunately, in some cases, the bioactive agent or agents included in the composition may be irritative or damaging to the eye or epithelial surface (e.g., cyclosporin A). Advantageously, through its rheological property and other properties, the high molecular weight HA in the composition can alleviate and/or protect the epithelium such as epithelium of the eye from the irritative and/or damaging effects of the biologically active agent or agents within the composition (i.e., the bioactive agent would be more irritative or more damaging to the epithelial surface if administered without the high molecular weight HA).

In some embodiments, the bioactive agent included in the HMW HA composition is an immunomodulatory agent. The HMW HA can make the environment at the epithelial surface more conducive to the immunomodulatory agent's activity, enhancing or facilitating the action of the immunomodulatory agent.

In some embodiments, the composition contains no steroid, antibiotic, or immunomodulator. In some embodiments, the composition contains no other bioactive agent (e.g., no hydrophobic active ingredient).

In some circumstances, it may be desirable to include one or more preservatives or detergents within the composition. Often, such preservatives and detergents are irritative or damaging to the epithelium, such as ocular epithelium. Advantageously, through its rheological property and other properties, the composition can alleviate and/or protect the epithelium from the irritative and/or damaging effects of the preservative or detergent within the composition. Thus, in some embodiments, the composition further comprises a preservative or detergent that is irritative or damaging to the epithelium such as ocular epithelium (i.e., a preservative or detergent that would be more irritative or more damaging to the epithelium if administered without the high molecular weight HA). In some embodiments, the composition contains no preservative or detergent.

In some embodiments, the composition includes cyclosporin A, cetalkoniumchloride, tyloxapol, or a combination of two or more of the foregoing.

In some embodiments, the composition includes a bioactive agent that is an immunosuppressant (e.g., a T-cell inhibitor such as cyclosporin A, tacrolimus, or sirolimus; antimetabolite; alkylating agent; TNF inhibitor (e.g., infliximab, etanercept, or adilumab); lymphocyte inhibitor; or interleukin inhibitor); a prostaglandin (e.g., latanoprost), prostaglandin analog, or other agent that reduces intraocular pressure; an antihistamine and/or mast cell stabilizer (e.g., ketotifen), or a combination of two or more of the foregoing. The epithelial surface to which the composition is topically administered may be an ocular surface or a non-ocular surface. The composition may further include a preservative and/or detergent, or lack any preservative or detergent.

In some embodiments, the composition includes a bioactive agent that is an anti-inflammatory agent selected from among a glucocorticoid or other steroid (e.g., prednisone, cortisone acetate, prednisolone, methylprednisolone, dexamethasone, betamethasone, triamcino lone, beclomethasone, fludrocortisone acetate, deoxycorticosterone acetate, aldosterone), non-steroidal anti-inflammatory drug (e.g., salicylates, arylalkanoic acids, 2-arylpropionic acids, N-arylanthranilic acids, oxicams, coxibs, or sulphonanilides), Cox-2-specific inhibitor (e.g., valdecoxib, celecoxib, or rofecoxib), leflunomide, gold thioglucose, gold thiomalate, aurofin, sulfasalazine, hydroxychloroquinine, minocycline, TNF-alpha binding protein (e.g., infliximab, etanercept, or adalimumab), abatacept, anakinra, interferon-beta, interferon-gamma, interleukin-2, allergy vaccine, antihistamine, antileukotriene, beta-agonists, theophylline, or anticholinergic, antibiotic, tacrolimus, or retinoid.

In some embodiments, the composition includes one or more of a solvent, co-solvent, demulcent, emollient, preservative, antioxidant, moisturizer, or solubilizing agent.

In some embodiments, the composition is administered to the subject before, during, and/or after administration of another composition comprising a bioactive agent to the subject. In some circumstances, it may be desirable to include one or more preservatives or detergents within the other composition. As indicated above, often, such preservatives and detergents are irritative or damaging to the eye, and some bioactive agents themselves may be irritative or damaging to the eye. Advantageously, through its rheological property and other properties, the composition can alleviate and/or protect the eye from the irritative and/or damaging effects of the bioactive agent, preservative, and/or detergent within the other composition. Thus, the bioactive agent, preservative, and/or detergent within the other composition would be more irritative or more damaging to the epithelium, such as that of the eye, if administered without the composition.

In some embodiments, the other composition that is administered before, during, or after the HMW HA composition includes an immunomodulatory agent. The HMW HA can make the environment at the epithelial surface more conducive to the immunomodulatory agent's activity, enhancing or facilitating the action of the immunomodulatory agent.

In some embodiments, the other composition includes cyclosporin A, cetalkoniumchloride, tyloxapol, or a combination of two or more of the foregoing.

In some embodiments, the epithelial surface is an ocular surface, and the other composition is an immunosuppressant (e.g., a T-cell inhibitor such as cyclosporin A, tacrolimus, or sirolimus; antimetabolite; alkylating agent; TNF inhibitor (e.g., infliximab, etanercept, or adilumab); lymphocyte inhibitor; or interleukin inhibitor), a prostaglandin (e.g., latanoprost); prostaglandin analog or other agent that reduces intraocular pressure; an antihistamine and/or mast cell stabilizer (e.g., ketotifen), or a combination of two or more of the foregoing. The other composition may further include a preservative and/or detergent, or lack any preservative or detergent.

The other composition administered to the subject may be in any form and administered by any route (e.g., local or systemic). In some embodiments, the other composition is administered to the eye, e.g., topically or by injection. In some embodiments, the other composition is topically administered to the ocular surface.

In some embodiments, the preservative or detergent included in the composition is a chemical preservative or oxidative preservative.

In some embodiments, the preservative or detergent included in the composition is one that kills susceptible microbial cells by disrupting the lipid structure of the microbial cell membrane, thereby increasing microbial cell membrane permeability.

In some embodiments, the preservative or detergent included in the composition is one that causes damage to the corneal tissues, such as corneal epithelium, endothelium, stroma, and interfaces such as membranes, in the absence of the administered HMW HA.

In some embodiments, the preservative or detergent included in the fluid or other composition is selected from the group consisting of quaternary ammonium preservative (e.g., benzalkonium chloride (BAK) or cetalkoniumchloride), chlorobutanol, edetate disodium (EDTA), polyquaternarium-1 (e.g., Polyquad™ preservative), stabilized oxidizing agent (e.g., stabilized oxychloro complex (e.g., Purite™ preservative)), ionic-buffered preservative (e.g., sofZia™ preservative), polyhexamethylene biguanide (PHMB), sodium perborate (e.g., GenAqua™ preservative), tylopaxol, and sorbate.

The composition may be a health and beauty product, such as one selected from a bath and shower product, baby and kid care product, sun care product, shampoo, hair condition, body lotion, solid or liquid soap, hair styling product, shaving cream, after shave product, hand and nail cream, face cream, face cleansing lotion, cologne, mouthwash, or toothpaste. In some embodiments, composition is a cosmetic product, such as a foundation, mascara, eye shadow, eye liner, peel, scrub, face mask, skin tightener, toner, body butter, sun care product, shower gel, eye cream, hand and cuticle cream, or makeup powder.

The composition may be applied by hand or an applicator, depending upon the epithelial surface. In some embodiments, the composition is administered using an applicator, such as a dropper, swab, cosmetic pad, wipe, wipe stick, towelette, sponge, gauze, puff, wand, adhesive or non-adhesive bandage or contact lens.

In some embodiments, the composition is at least essentially mucin-free; or in other words having a mucin concentration of <0.3% w/v.

In some embodiments, the composition includes a preservative. In other embodiments, the fluid does not include a preservative (i.e., the fluid is preservative-free).

In some embodiments, the composition further includes a glycosaminoglycan (GAG), i.e., includes one or more GAGs in addition to the high molecular weight HA; electrolyte (e.g., sodium chloride); buffer (e.g., phosphate buffer); or a combination of two or more of the foregoing.

The subject may or may not have dry eye syndrome (the aqueous tear deficiency type or qualitative dry eye type) at the time the composition is administered to the eye of the subject. In some embodiments of the therapeutic or prophylactic methods, the condition is an irritation, discomfort, inflammation, immune response, or combination of two or more of the foregoing, at an ocular surface, and the eye of the subject to which the fluid is administered does not have aqueous tear deficiency (ATD) at the time of administering the fluid (i.e., in the absence of ATD). In some embodiments of the therapeutic or prophylactic methods, the condition is an irritation, discomfort, inflammation, immune response, or combination of two or more of the foregoing, at an ocular surface, and the eye of the subject to which the fluid is administered does not have qualitative dry eye at the time of administering the fluid (i.e., in the absence of qualitative dry eye). In some embodiments of the therapeutic or prophylactic methods, the condition is an irritation, discomfort, inflammation, immune response, or combination of two or more of the foregoing, at an ocular surface, and the eye of the subject to which the fluid is administered does not have dry eye syndrome at the time of administering the fluid (i.e., in the absence of aqueous tear deficiency or qualitative dry eye).

In some embodiments of the therapeutic or prophylactic methods, the condition is an irritation, discomfort, inflammation, immune response, or combination of two or more of the foregoing, at an ocular surface, and the subject is not suffering from a tear volume deficiency; however, the subject has an ocular surface abnormality (a topographic anomaly) comprising elevations on the cornea or elsewhere on the eye surface for which the normal tear film (tear film of normal surface tension and viscosity) does not cover, resulting in areas of friction at the ocular surface.

The fluid may be used in conjunction with a bandage contact lens. Thus, the method may further include applying a bandage contact lens to the eye before, during, and/or after administering the fluid. For example, the fluid may be administered before applying the bandage contact lens, after the contact lens, and/or placing fluid on the bandage contact lens before applying the bandage contact lens to the eye. Use of the fluid allows the bandage contact lens to exert pressure on the ocular surface while simultaneously minimizing friction at the ocular surface. Advantageously, the fluid and bandage contact lens can safely be used shortly after ocular surgery, e.g., glaucoma surgery.

Another aspect of the invention is a kit comprising the HMW HA composition for carrying out the methods of the invention. Optionally, the kit includes an applicator for applying the composition to the desired epithelial surface.

Optionally, the kit further comprises a container containing the composition. Such as a bottle, tube, vase, or sachet.

The applicator may be pretreated with, or contains, the composition.

In some embodiments, the applicator is a swab, cosmetic pad, wipe, wipe stick, towelette, sponge, gauze, puff, wand, brush, comb, dropper, or adhesive or non-adhesive bandage.

The container of the kit may include a closure selected from a pump, spray, or cap.

In some embodiments, the kit further comprises instructions for applying the composition to an epithelial surface by topically administering the composition to the epithelial surface, and optionally instructions for restoring hyaluronic acid to an epithelial surface having a deficiency in the amount or function of hyaluronic acid.

In another embodiment, the kit comprises the composition described herein as a fluid, and one or more bandage contact lenses. Bandage contact lenses may be packaged together with the composition within the same container (with the bandage contact lenses in contact with the composition), or the bandage contact lenses may be separate from the fluid, packaged in separate containers. Suitable containers include, for example, bottles, vials, syringes, blister pack, etc. The containers may be formed from a variety of materials such as glass or plastic.

The kit may include a delivery agent (separately or in association with the fluid) that is to be brought into contact with the ocular surface or other part of the eye. For example, the kit may include particles (e.g., microparticles or nanoparticles) that are coated with the fluid and/or release the fluid onto the ocular surface.

Optionally, the kit may include an applicator or device for dispensing eye drops (e.g., an eye dropper), which may or may not serve as a container for the fluid in the kit before the kit's outer packaging is accessed (e.g., opened), i.e., the eye drop dispensing device may function to contain the fluid provided in the unaccessed (unopened) kit, or may be empty and receive the fluid after the kit is accessed. Optionally, the kit may include a label or packaging insert with printed or digital instructions for use of the kit, e.g., for carrying out the method of the invention.

Kits of the invention can include packaging material that is compartmentalized to receive one or more containers such as vials, tubes, and the like, each of the container(s) including one of the separate elements to be used in a method described herein. Packaging materials for use in packaging pharmaceutical products include, by way of example only U.S. Pat. Nos. 5,323,907, 5,052,558 and 5,033,252. Examples of pharmaceutical packaging materials include, but are not limited to, blister packs, bottles, tubes, pumps, bags, vials, light-tight sealed containers, syringes, bottles, and any packaging material suitable for a selected formulation and intended mode of administration and treatment.

A kit may include one or more additional containers, each with one or more of various materials desirable from a commercial and user standpoint for use of the compositions described herein. Non-limiting examples of such materials include, but not limited to, buffers, diluents, carrier, package, container, vial and/or tube labels listing contents and/or instructions for use, and package inserts with instructions for use.

A label can be on or associated with the container. A label can be on a container when letters, numbers or other characters forming the label are attached, molded or etched into the container itself; a label can be associated with a container when it is present within a receptacle or carrier that also holds the container, e.g., as a package insert. A label can be used to indicate that the contents are to be used for a specific therapeutic application. The label can also indicate directions for use of the contents, such as in the methods described herein.

In some embodiments of the kit, the fluid can be presented in a pack or dispenser device which can contain one or more unit dosage forms containing a composition disclosed herein. The pack can for example contain metal or plastic foil, such as a blister pack. The pack or dispenser device can be accompanied by instructions for administration.

### Fluid Preparation

As indicated above, the HA used in the compositions and methods of the invention is high molecular weight (HMW). In some embodiments, the hyaluronic acid of the fluid has an intrinsic viscosity of greater than 2.5 m³/kg. In some embodiments, the hyaluronic acid has an intrinsic viscosity of greater than 2.9 m³/kg.

Furthermore, in cases in which the epithelial surface is epithelium of the ocular surface, the concentration of HMW HA and/or HMW HA analogue is preferably <0.2% w/v. In other cases, in which the epithelial surface is epithelium of the skin, for example, the concentration of HMW HA and/or HMW HA analogue is preferably within the range of 0.2% to 3.0% w/v.

Viscoelasticity is defined as characteristics of a fluid having both viscous and elastic properties. The zero shear viscosity is determined as the steady shear plateau viscosity at vanishing shear rate. For highly viscous formulations, measurement with a controlled stress rheometer is preferred.

The relation between molecular weight and intrinsic viscosity [η] in m³/kg is given through the Mark-Houwink equation:

[η]=k·(Mᵣₘ)^{a}

with Mᵣₘ being the molecular mass in MDa and the coefficients

k=1.3327·10⁻⁴

and

a=0.6691

which values for k and a having been found as most predictive.

The fluid may be produced by: sterilizing the filling line if desired; adding purified water or water for injection (WFI) to a stainless steel mixing tank; adding salts while mixing; slowly adding HA and mixing until a homogeneous solution/fluid is achieved; adjusting pH value by adding NaOH or HCl, if required, while continuing the mixing process; transferring the solution over a 1 μm pore size filter cartridge to a sterile holding tank; and aseptically filling the solution via sterile filtration into the sterile primary package (monodose or vial). In the case of monodoses, this may be done by a blow-fill-seal (BFS) process. For ocular applications, the composition is preferably sterile. For applications such as skin, for example, the composition may be sterile but not necessarily.

Preferably, the fluid is at least essentially mucin-free or in other words having a mucin concentration of <0.3% w/v. This means that the flow behavior or properties essentially is reached or adjusted by hyaluronan and not by mucin naturally present in the subject's tear fluid and mainly responsible for the flow behavior thereof.

It is preferred that if substances are added that increase the viscosity, they are added towards, or during, or as a final step. The mixing is carried out so as to reach a homogeneous mixture. As an alternative or in addition, it is preferred to initially provide purified water or water for injection as a basis, and then, optionally, electrolytes, buffers and substances which do not increase the viscosity are added at first to the purified water or water for injection.

HA is further described in the monograph of the European Pharmacopoeia 9.0, page 3583 (Sodium Hyaluronate), which is incorporated herein by reference in its entirety.

In one embodiment, the fluid used in the method and kit of the invention has the characteristics listed in Table 1:

TABLE 1

| Characteristic | Specification | Test Method |
|---|---|---|
| Appearance | clear and colorless solution, | Ph.Eur. |
| | free from visible impurities | |
| pH value | 6.8-7.6 | Ph.Eur. |
| Osmolality | 240-330 mosmol/kg | Ph.Eur. |
| HA concentration | 0.10-0.19% w/v | Ph.Eur. |
| NaCl concentration | 7.6-10.5 g/l | Ph.Eur. |
| Sterility | Sterile | Ph.Eur. |
| Phosphate concentration | 1.0-1.4 mmol/l | Ph.Eur. |

### Definitions

The term “a,” “an,” “the” and similar terms used in the context of the present invention (especially in the context of the claims) are to be construed to cover both the singular and plural unless otherwise indicated herein or clearly contradicted by the context. Thus, for example, reference “a cell” or “a compound” should be construed to cover both a singular cell or singular compound and a plurality of cells and a plurality of compounds unless indicated otherwise or clearly contradicted by the context. Similarly, the word “or” is intended to include “and” unless the context clearly indicates otherwise. The abbreviation, “e.g.” is derived from the Latin exempli gratia, and is used herein to indicate a non-limiting example. Thus, the abbreviation “e.g.” is synonymous with the term “for example.”

The term “deficiency”, in the context of HA at an epithelial surface, means lacking in the amount or structure of HA (membrane-bound HA or unbound HA) relative to what is physiologically normal, or lacking in one or more biological functions of HA that are normal for the extracellular space within the epithelium and the tissues immediately beneath, at the anatomical location. Methods for the determination of HA content and size in vivo are known (Cowman M K, “Hyaluronan and Hyaluronan Fragments”, Adv Carbohydr Chem Biochem., 2017, 74:1-59, and particularly Chapter 6: Experimental Determination of HA Content and Size In Vivo, which is incorporated herein by reference in its entirety).

The phrase “effective amount”, in the context of the administered composition, refers to the amount of the composition necessary to obtain a desired result, such as restoration (full or partial) of HA or one or more biological functions of HA at the desired anatomical site. In some embodiments, an effective amount may be the amount capable of preventing, delaying the onset of, treating, or ameliorating a disease or condition, or otherwise capable of producing an intended therapeutic effect.

The term “epithelial surface” means a surface of the body composed mainly of epithelial cells. Epithelia are sheets of cells that cover most of the body's surfaces. Epithelia are a group of tissues, perform a wide variety of functions, and adopt different cellular arrangements and structure to accomplish these functions. The epithelium may be any type of epithelium. It may be keratinized (cornified), such as skin, or non-keratinized, such as the mouth, esophagus, and vagina. For example, the epithelium may be simple squamous epithelium (e.g., air sacs of lungs, and lining of the heart, blood vessels, and lymphatic vessels), simple cuboidal epithelium (e.g., in ducts and secretory portions of small glands and in kidney tubules), simple columnar epithelium (e.g., ciliated tissues in bronchi, uterine tubes, and uterus; smooth (non-ciliated) tissues in digestive tract and bladder), pseudostratified columnar epithelium (e.g., ciliated tissue lining the trachea and much of the upper respiratory tract), stratified squamous epithelium (e.g., lining the esophagus, mouth, and vagina), stratified cuboidal epithelium (e.g., sweat glands, salivary glands, mammary glands), stratified columnar epithelium (e.g., male urethra and ducts of some glands), transitional epithelium (e.g., lining the bladder, urethra, and ureters), or any combination of two or more of the foregoing.

Epithelial cells are normally characterized by the presence of tight junctions, adhering junctions, and desmosomes. Epithelial cells are typically polarized with an apical surface facing a lumen or external environment, and a basal surface facing the basement membrane. The composition may be topically administered to an apical surface, or to a basal surface if the basal surface is exposed. The epithelial surface may be intact or continuous, or discontinuous, such as resulting from trauma.

The phrase “biological function of hyaluronic acid” means one or more functions of biological HA in the tissue in question, such as epithelium. For example, depending upon the anatomical site of the epithelium, a function of HA may include one or more of: hydration, water binding of HA within the extracellular matrix (enabling transport of nutrients, catabolites, and gasses, thereby nourishing tissues), lubrication, space-filling capacity, framework through which cells migrate, tissue viscosity, shock absorption, free-radical scavenging, cytokine interaction, and modulation of each of inflammation, cell migration, proliferation, and differentiation via HA receptors (see, for example, Dicker K T et al., “Hyaluronan: A Simple Polysaccharide with Diverse Biological Functions”, Acta Biomater, 2014, 10(4): 1558-1570)).

The term “isolated,” when used as a modifier of a composition, means that the compositions are made by human intervention or are separated from their naturally occurring in vivo environment. Generally, compositions so separated are substantially free of one or more materials with which they normally associate with in nature, for example, one or more protein, nucleic acid, lipid, carbohydrate, cell membrane. A “substantially pure” molecule can be combined with one or more other molecules. Thus, the term “substantially pure” does not exclude combinations of compositions. Substantial purity can be at least about 60% or more of the molecule by mass. Purity can also be about 70% or 80% or more, and can be greater, for example, 90% or more. Purity can be determined by any appropriate method, including, for example, UV spectroscopy, chromatography (e.g., HPLC, gas phase), gel electrophoresis (e.g., silver or coomassie staining) and sequence analysis (for nucleic acid and peptide).

As used herein, the term “homeostasis” refers to the capacity of a physiological system to maintain internal stability, or to the state of stability itself, owing to the coordinated response of its parts to any situation or stimulus that would tend to disturb its normal, non-pathological condition or function.

As used herein, the term “hyaluronic acid” or “HA” refers to the glycosaminoglycan composed of disaccharide repeats of N-acetylglucosamine and glucuronic acid found in nature, also known as hyaluronan (e.g., the straight chain, glycosaminoglycan polymer composed of repeating units of the disaccharide [-D-glucuronic acid-b1,3-N-acetyl-D-glucosamine-b1,4-]n), as well as derivatives of hyaluronan having chemical modifications such as esters of hyaluronan, amide derivatives, alkyl-amine derivatives, low molecular weight and high molecular weight forms of hyaluronans, and cross-linked forms such as hylans. Thus, the disaccharide chain may be linear or non-linear. Hyaluronan can be cross-linked by attaching cross-linkers such as thiols, methacrylates, hexadecylamides, and tyramines. Hyaluronan can also be cross-linked directly with formaldehyde and divinylsulfone. Examples of hylans include, but are not limited to, hylan A, hylan A (a formaldehyde cross-linked glycosaminoglycan polymer), hylan B (a divinylsulfone cross-linked glycosaminoglycan polymer), and hylan G-F 20 (Cowman M K et al., Carbohydrate Polymers 2000, 41:229-235; Takigami S et al., Carbohydrate Polymers, 1993, 22:153-160; Balazs E A et al., “Hyaluronan, its cross-linked derivative—Hylan—and their medical applications”, in Cellulosics Utilization: Research and Rewards in Cellulosics, Proceedings of Nisshinbo International Conference on Cellulosics Utilization in the Near Future (Eds Inagaki, H and Phillips G O), Elsevier Applied Science (1989), NY, pp. 233-241; Koehler L et al., Scientific Reports, 2017, 7, article no. 1210; and Pavan M et al., Carbohydr Polym, 2013, 97(2): 321-326; which are each incorporated herein by reference in their entirety).

The term “hyaluronic acid” or HA includes HA itself and pharmaceutically acceptable salts thereof. The HA can be formulated into pharmaceutically-acceptable salt forms. Pharmaceutically-acceptable salts of HA can be prepared using conventional techniques.

The term “high molecular weight” or “HMW” in the context of HA of the invention refers to hyaluronic acid having an intrinsic viscosity of >2.5 m³/kg as determined by the method of the European Pharmacopoeia 9.0, “Sodium Hyaluronate”, page 3584 (which is incorporated herein by reference in its entirety). Briefly, the intrinsic viscosity [η] is calculated by linear least-squares regression analysis using the Martin equation: Log₁₀(nᵣ−1/c)=log₁₀[η]+κ[η]c. In some embodiments, the high molecular weight hyaluronic acid has an intrinsic viscosity of greater than 2.9 m³/kg.

As used herein, the term “immunocompromised” refers to a subject with an innate, acquired, or induced inability to develop a normal immune response. An immunocompromised subject, therefore, has a weakened or impaired immune system relative to one of a normal subject. A subject with a weakened or impaired immune system has an “immunodeficiency” or “immunocompromised condition,” which is associated with a primary or secondary deficiency, induced or non-induced, in one or more of the elements of the normal immune defense system. An immunocompromised condition may be due to a medical treatment, e.g., radiation therapy, chemotherapy or other immunosuppressing treatment, such as induced by treatment with steroids, cyclophosphamide, azathioprine, methotrexate, cyclosporine or rapamycin, in particular in relation to cancer treatment or the treatment or prevention of transplant rejection. The presence of an immunocompromised condition in a subject can be diagnosed by any suitable technique known to persons of skill in the art. Strong indicators that an immunocompromised condition may be present are when rare diseases occur or the subject gets ill from organisms that do not normally cause diseases, especially if the subject gets repeatedly infected. Other possibilities are typically considered, such as recently acquired infections, for example, HIV, hepatitis, tuberculosis, etc. Generally, however, definitive diagnoses are based on laboratory tests that determine the exact nature of the immunocompromised condition. Most tests are performed on blood samples. Blood contains antibodies, lymphocytes, phagocytes, and complement components, all of the major immune components that might cause immunodeficiency. A blood cell count can be used to determine if the number of phagocytic cells or lymphocytes is below normal. Lower than normal counts of either of these two cell types correlates with an immunocompromised condition. The blood cells are also typically checked for their appearance. Sometimes, a subject may have normal cell counts, but the cells are structurally defective. If the lymphocyte cell count is low, further testing is usually conducted to determine whether any particular type of lymphocyte is lower than normal. A lymphocyte proliferation test may be conducted to determine if the lymphocytes can respond to stimuli. The failure to respond to stimulants correlates with an immunocompromised condition. Antibody levels and complement levels can also be determined for diagnosing the presence of an immunocompromised condition.

As used herein, the term “ocular surface” refers to the structures of the eye and adnexa, including the cornea, conjunctiva, eyelids, eyelashes, tear film, main and accessory lacrimal glands, and the meibomian glands. Thus, the tears, both in terms of the individual components at the site of production, and as a film on the ocular surface, are included with the term “ocular surface” (see Craig J P et al., “TFOS DEWS II Definition and Classification Report”, The Ocular Surface, 2017, 15:276-283, which is incorporated herein by reference in its entirety). The composition may be topically administered to one or more parts of the ocular surface, including, for example, the entire ocular surface.

“Pharmaceutically acceptable salt” includes both acid and base addition salts. A pharmaceutically acceptable salt of HA or any one of the other compounds described herein is intended to encompass any and all pharmaceutically suitable salt forms. Preferred pharmaceutically acceptable salts described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997), which is hereby incorporated by reference in its entirety). Acid addition salts of basic compounds may be prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts may be formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra. In some embodiments, the pharmaceutically acceptable salt is sodium salt (see “Sodium Hyaluronate” at page 3583 of European Pharmacopoeia 9.0, which is incorporated herein by reference).

As used herein, the terms “subject”, “patient”, and “individual” refer to a human or non-human animal. A subject also refers to, for example, primates (e.g., humans), cows, sheep, goats, horses, dogs, cats, rabbits, rats, mice, fish, birds and the like. In some embodiments, the subject is a mammal. In some embodiments, the subject is a human. In some embodiments, the subject is a bird or fish. Thus, the methods may be carried out in the medical setting and the veterinary setting. The non-human animal subject may be, for example, a pet or an animal model of an ocular or non-ocular disease. The subject may be any age or life stage. For example, in some embodiments, the subject is an infant or adolescent. In some other embodiments, the subject is elderly. In some embodiments, the subject is atopic (has atopy).

In some embodiments, the eye of the subject does not have aqueous tear deficiency (ATD) at the time of said administering (i.e., in the absence of ATD).

In some embodiments, the eye of the subject does not have qualitative dry eye at the time of said administering (i.e., in the absence of qualitative dry eye).

In some embodiments, the eye of the subject does not have dry eye syndrome at the time of said administering (i.e., in the absence of aqueous tear deficiency or qualitative dry eye).

In some embodiments, the subject is not suffering from an aqueous tear deficiency (ATD), and wherein the subject has an ocular surface abnormality (a topographic anomaly) comprising elevations on the cornea or elsewhere on the eye surface for which the normal tear film (tear film of normal surface tension and viscosity) does not cover, resulting in areas of friction at the ocular surface, and wherein the administered fluid reduces the friction.

In some embodiments, the subject is immunocompromised, i.e., is in an immunocompromised condition.

The phrase “topical administration” is used herein in its conventional sense to mean topical delivery to the desired anatomical site, such as an epithelial surface (a surface of epithelium). The composition comprising HMW HA may be applied directly or indirectly to the epithelial surface by any manner that allows an effective amount of the composition and the epithelial surface to make contact. For example, the composition may be applied directly to the epithelial surface, such as via eye drops or lavage, or applied indirectly via a delivery agent that is brought into contact with the epithelial surface. An example of a delivery agent is a particle (e.g., microparticles or nanoparticles) that is coated with the composition and/or releases the composition onto the ocular surface. Such particles may be composed of various materials, such as natural or synthetic polymers. In some embodiments, the delivery agent may itself be administered as drops.

The invention is described only exemplarily by the embodiments in the description and drawings and is not limited thereto but rather includes all variations, modifications, substitutions, and combinations the expert may take from the complete documents of this application under consideration of and/or combination with his specific knowledge.

All patents, patent applications, provisional applications, and publications referred to or cited herein are incorporated by reference in their entirety, including all figures and tables, to the extent they are not inconsistent with the explicit teachings of this specification.

Following is an example that illustrates procedures for practicing the invention. These examples should not be construed as limiting. All percentages are by weight and all solvent mixture proportions are by volume unless otherwise noted.

### EXAMPLE 1

### Multi-Center, Multi-National Prospective Clinical Study on Patients with Severe Dry Eyes—The HYLAN M Study

A multi-center, multi-national prospective, randomized clinical study on patients with severe dry eyes (according to the ODISSEY primary criteria) is being conducted in 12 study centers in 9 countries. Within the HYLAN M study, the patients are randomized in two groups, one staying with the most effective individual patient treatment identified before, the other one switched to high molecular weight hyaluronic acid eye drops (Comfort Shield® preservative-free sodium hyaluronate eye drops (i.com medical GmbH, Munich, Germany)), which corresponds to the embodiment of Table 1 herein.

These patients (192 enrolled) have already received the best treatment their ophthalmologists could offer. All of the patients had been under “stable” therapy at the time of their inclusion into the study, i.e., their therapy has not been changed over a defined period of time prior to inclusion into this study. The patients are randomized into two groups, one group of patients remaining with their current therapy for dry eye syndrome, and the second group of patients treated with drops of the fluid described above (Comfort Shield® eye drops) in place of their tear substitute.

Study objectives include: (1) comparison of objective and subjective symptoms of dry eye under treatment with Comfort Shield® eye drops versus the tear substitute eye drops which the patients has been treated with before presenting to the investigator (=current therapy) in severe dry eye conditions; and (2) to observe objective performance, patients' subjective acceptance and adverse events of the eye drops. For each patient, both eyes are examined, and the eye with higher corneal fluorescein staining score at baseline examination is evaluated.

The patients in one of the study centers involved in the HYLAN M study had thus far not achieved adequate relief of signs and symptoms by all commercially available eye drops that the physicians had tested before deciding in favor of the treatment with autologous serum eye drops. They have included 11 patients with autologous serum eye drop treatment into the study. Out of these 11 patients, 6 have been randomized to the Comfort Shield® group, i.e. the use of autologous serum eye drops was replaced by Comfort Shield® over a period of 8 weeks. Out of these 6 patients, 2 discontinued their participation in the study, because Comfort Shield® eye drops did not provide adequate relief of symptoms. Two continued with the Comfort Shield® eye drop therapy over the eight weeks of the study, but preferred to return to their original therapy with autologous serum. The remaining two patients preferred Comfort Shield® eye drops over the autologous serum eye drops and decided to use the Comfort Shield® eye drops beyond the study.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

1. A method for restoring or replenishing hyaluronic acid at an epithelial surface of a subject, comprising topically administering a composition to the epithelial surface of the subject, wherein the composition comprises high molecular weight hyaluronic acid, high molecular weight hyaluronic acid analogue, or a combination thereof, and wherein the epithelial surface has a deficiency in the amount or function of hyaluronic acid.

2. The method of claim 1, wherein the epithelial surface is a site of a hypersensitivity reaction in the subject, and the composition is topically administered to the epithelial surface prophylactically before onset of the hypersensitivity rejection.

3. The method of claim 1, wherein the epithelial surface is a site of a hypersensitivity reaction in the subject, and the composition is topically administered to the epithelial surface therapeutically after onset of the hypersensitivity rejection.

4-5. (canceled)

6. The method of claim 1, wherein the subject has atopy.

7. The method of claim 1, wherein the epithelial surface is on the interior of the body.

8. The method of claim 1, wherein the epithelial surface lines the interior of a body cavity.

9. The method of claim 1, wherein the epithelial surface on the exterior of the body.

10. The method of claim 1, wherein the epithelial surface is epithelium of the skin.

11. The method of claim 1, wherein the epithelial surface is the surface of a mucous membrane.

12. (canceled)

13. The method of claim 1, wherein the epithelial surface is an ocular surface.

14. The method of claim 1, wherein the epithelial surface is a non-ocular surface.

15-18. (canceled)

19. The method of claim 1, wherein the composition contains no other bioactive agent.

20-47. (canceled)

48. The method of claim 1, wherein the high molecular weight hyaluronic acid and/or high molecular weight hyaluronic acid analogue has an intrinsic viscosity of >2.5 m³/kg.

49. The method of claim 1, wherein the high molecular weight hyaluronic acid and/or high molecular weight hyaluronic acid analogue has an intrinsic viscosity of >2.9 m³/kg.

50. The method of claim 1, wherein the epithelial surface comprises an ocular surface, and wherein the composition has a concentration of <0.2% w/v high molecular weight hyaluronic acid and/or high molecular weight hyaluronic acid analogue.

51. The method of claim 1, wherein the epithelial surface comprises skin, and wherein the composition has a concentration of 0.2% to 3.0% w/v high molecular weight hyaluronic acid and/or high molecular weight hyaluronic acid analogue.

52-53. (canceled)

54. The method of claim 3, wherein the hypersensitivity reaction is associated with an atopic disorder selected from among: conjunctivitis, rhinoconjunctivitis, intrinsic atopic dermatitis (i.e., non-IgE-associated), extrinsic atopic dermatitis (i.e., IgE-associated eczema), immune-mediate urticaria, immune-mediated angiodema, acute (IgE-mediated) latex allergy, allergic asthma, IgE-mediated components of allergic bronchopulmonary aspergillosis, allergic rhinitis (hay fever), eosinophilic gastroenteritis (EGE), and eosinophilic colitis (ECO).

55-59. (canceled)

60. The method of claim 1, wherein the subject has an eye with keratoconus, and wherein the composition is topically administered to an ocular surface of the eye.

61-68. (canceled)

69. An applicator pre-treated with, or containing, the composition of claim 1.

70. (canceled)

71. A kit comprising the composition of claim 1.

72-78. (canceled)
